(19) United States
(12) Patent Application Publication
(10) Pub. No.: US 2025/0218603 A1
(43) Pub. Date: Jul. 3, 2025

(54) CASE MANAGEMENT PLATFORM

(71) Applicants: Eddie Brodie, Boston, MA (US); Joseph Broady, Boston, MA (US)

(72) Inventors: Eddie Brodie, Boston, MA (US); Joseph Broady, Boston, MA (US)

(21) Appl. No.: 19/003,681

(22) Filed: Dec. 27, 2024

Related U.S. Application Data
(60) Provisional application No. 63/615,142, filed on Dec. 27, 2023.

(51) Int. Cl. G16H 80/00; G16H 10/60; G16H 40/20

(52) U.S. Cl. CPC G16H 80/00; G16H 10/60; G16H 40/20

(57) ABSTRACT

A case management platform may connect to communication sources and software systems to monitor for notifications. The platform may receive a notification comprising a communication from a stakeholder or an event notification from a software system. The platform may parse the notification to determine relevant information for performing actions. Based on the information, the platform may cause actions to be performed. The actions may comprise executing operations in a software system, transmitting a synthesized voice communication to stakeholders associated with the notification, or storing records associated with the notification to a database. The platform may include hardware processors and non-transitory computer-readable storage media storing instructions to perform the operations.

### RELATED APPLICATION(S)

Under provisions of 35 U.S.C. § 119 (e), the Applicant(s) claim the benefit of U.S. Provisional Application No. 63/615,142 filed Dec. 27, 2023, which is incorporated herein by reference.

It is intended that each of the referenced applications may be applicable to the concepts and embodiments disclosed herein, even if such concepts and embodiments are disclosed in the referenced applications with different limitations and configurations and described using different examples and terminology.

### FIELD OF DISCLOSURE

The present disclosure generally relates to task coordination, and more specifically to a home care (e.g., including, but not limited to, home healthcare) agency and/or case management coordination.

### BACKGROUND

Home care agencies, including (but not limited to) home healthcare agencies, provide supportive care in a client's home, Care may be provided by in-home workers, including licensed healthcare professionals who provide medical treatment needs, by professional and/or by any other professionals or paraprofessionals that assist clients with medical and non-medical treatments and activities. may also employ case managers or supervisors responsible for scheduling the in-home workers.

Home care agencies may serve a client base made up of adults, seniors, and/or pediatric clients who are recovering after a hospital or facility stay, or need additional support to remain safely at home and avoid unnecessary hospitalization. The services provided to clients may include short-term nursing, rehabilitative, therapeutic, and/or assistive home health care. This care may be provided by registered nurses (RNs), licensed practical nurses (LPN's), physical therapists (PTs), occupational therapists (OTs), speech language pathologists (SLPs), unlicensed assistive personnel (UAPs), home health aides (HHAs), medical social workers (MSWs) and/or other professional caretakers. However, the largest and most venerable segment of the home care population involves assisting individuals with non-medical daily tasks such as (but not limited to) bathing, dressing, eating, and moving about the home (e.g., transferring in and out of bed). Such help is carried out by caregivers or other licensed or unlicensed non-medical personnel.

Conventional systems in the home care industry may rely heavily on manual processes and human intervention for case management. These systems may require case managers to manually schedule in-home workers, track caregiver hours, manage client records, and coordinate care. Communication between stakeholders may be fragmented across multiple channels, potentially leading to inefficiencies and delays in information sharing. Traditional case management approaches may limit the number of clients a single case manager can effectively oversee, potentially constraining agency growth and scalability. Compliance tracking and crisis management may be challenging to manage in real-time with conventional methods. Additionally, existing systems may struggle to provide multilingual support for diverse client populations, potentially creating language barriers in care delivery. Manual documentation processes may be time-consuming and prone to errors, potentially impacting the quality and consistency of care records. Furthermore, conventional systems may lack the ability to quickly analyze large amounts of data to identify trends or optimize care delivery.

Traditional case management systems for home care agencies require manual intervention and activities by a case manager to field large volumes of phone calls, each of which may require more follow-up phone calls, data entry via the case management system, and/or other follow-up actions by the case manager. These interactions create a bottleneck in productivity, as the case manager must contend with complex scheduling based on availability of the client, and the various in-home personnel. Currently, the case manager must manually enter/update information via common computer interface operations such as drag and drop, copy and paste, and/or direct data entry in the case management software system. The case manager has to perform these functions manually in the software system using a keyboard, mouse and/or finger/stylus on a computer, tablet or smartphone. Due to the volume and complexity of case manager calls, the home care agency's ability to fully service the personnel and client base in a timely and effective manner may be impacted unless headcount for case managers at the home care agency is increased. For tasks that do not require a case manager phone call, the case manager still must filter through the various screens, tabs, and/or widgets of the case management software system for scheduling, compliance, and/or client assessment functions to help ensure that clients have the proper coverage and receive adequate care. The manual nature of these tasks limits the productivity, or ability of the case manager to manage a particular client base.

The home care industry faces numerous challenges that may impact the quality and efficiency of care delivery. Traditional case management systems may struggle to handle the complex coordination required for effective home care services. These systems may lack the ability to quickly adapt to changing client needs or caregiver availability, potentially leading to gaps in care or suboptimal service delivery.

One technical problem in conventional home care management may be the difficulty in efficiently scheduling and coordinating caregivers across multiple clients with diverse needs. Manual scheduling processes may be time-consuming and prone to errors, potentially resulting in missed appointments or mismatched caregiver skills to client requirements. This may lead to decreased client satisfaction and increased administrative burden on case managers.

Another technical challenge may be the real-time tracking and management of caregiver compliance with regulatory requirements and client-specific care plans. Conventional systems may rely on periodic manual checks, which may not provide timely insights into potential compliance issues. This lag in information may increase the risk of non-compliance and potentially compromise the quality of care provided.

Effective communication among stakeholders, including caregivers, clients, family members, and healthcare providers, may present another technical hurdle. Traditional methods of communication, such as phone calls or emails, may not facilitate the rapid and secure exchange of information necessary for coordinated care. This may result in information silos, delayed decision-making, and potential miscommunications that could affect client outcomes.

Managing client data and maintaining comprehensive care records may pose additional technical challenges. Manual documentation processes may be prone to inconsistencies and may not provide a holistic view of a client's care history. This may hinder the ability of case managers to make informed decisions and may complicate the process of demonstrating compliance with regulatory requirements.

Crisis management and emergency response may be particularly challenging in conventional home care systems. The ability to quickly identify and respond to emergencies, such as client falls or sudden health deteriorations, may be limited by manual monitoring processes. This may result in delayed interventions and increased risk to client safety.

Scalability may be another significant technical problem faced by home care agencies. As agencies grow and serve larger client populations, traditional case management approaches may struggle to maintain efficiency and quality of care. The manual nature of many processes may limit the number of clients a single case manager can effectively oversee, potentially constraining agency growth or necessitating significant increases in staffing costs.

Multilingual support may present an additional technical challenge in diverse communities. Conventional systems may lack the capability to efficiently communicate with clients and caregivers in their preferred languages, potentially creating barriers to effective care delivery and client engagement.

Data analysis and trend identification may be difficult with traditional case management methods. The ability to quickly analyze large amounts of data to identify patterns in care delivery, client outcomes, or operational efficiency may be limited, potentially hindering continuous improvement efforts and strategic decision-making.

Lastly, ensuring the security and privacy of sensitive client information may pose significant technical challenges. Manual record-keeping and disparate communication channels may increase the risk of data breaches or unauthorized access to confidential health information.

Several currently available solutions attempt to address the challenges in home care management. Electronic health record (EHR) systems may provide digital documentation capabilities, potentially reducing manual paperwork. However, these systems may lack the specialized features required for home care coordination and may not integrate seamlessly with other necessary tools. Scheduling software may offer basic functionality for assigning caregivers to clients, but may struggle with complex scheduling scenarios or last-minute changes. Communication platforms designed for healthcare settings may facilitate information sharing among stakeholders, but may not provide the comprehensive case management features required for home care agencies.

These existing solutions may be inadequate for several reasons. EHR systems, while useful for maintaining client records, may not offer the specific functionality needed for home care coordination, such as caregiver scheduling, compliance tracking, or crisis management. They may also lack the ability to integrate with external communication channels commonly used in home care settings. Scheduling software may provide basic functionality but may struggle with the dynamic nature of home care, where client needs and caregiver availability can change rapidly. These systems may not offer the flexibility required to manage complex care plans or accommodate last-minute schedule adjustments.

Communication platforms designed for healthcare settings may improve information sharing but may not address the full spectrum of case management needs in home care. They may lack features for tracking caregiver compliance, managing authorized hours, or coordinating benefits. Additionally, these platforms may not provide the level of automation required to significantly increase case manager productivity or allow for the management of larger client pools.

Furthermore, existing solutions may not effectively leverage artificial intelligence or machine learning technologies to automate complex decision-making processes in case management. This limitation may result in continued reliance on manual intervention for many tasks, potentially limiting the scalability and efficiency of home care agencies. The inability to quickly analyze large amounts of data to identify trends or optimize care delivery may hinder continuous improvement efforts and strategic decision-making.

Multilingual support may be limited in current systems, potentially creating language barriers in diverse communities. This limitation may impact the quality of care delivery and client engagement, particularly in areas with multilingual populations. Additionally, existing solutions may struggle to provide real-time crisis management capabilities, potentially delaying response times to emergencies or sudden changes in client conditions.

In summary, while various solutions exist to address aspects of home care management, they may fall short in providing a comprehensive, integrated platform that can effectively handle the complex coordination, communication, and automation needs of modern home care agencies. The limitations of these existing solutions may contribute to ongoing challenges in efficiency, scalability, and quality of care delivery in the home care industry.

The home care industry faces significant challenges in managing the complex coordination, communication, and automation needs of modern agencies. There may be a need for a comprehensive, integrated solution that can effectively address these challenges while improving efficiency, scalability, and quality of care delivery. Existing solutions may fall short in providing the necessary features and capabilities to fully support the dynamic nature of home care, where client needs and caregiver availability can change rapidly. A more advanced approach may be required to overcome the limitations of current systems and enable home care agencies to better serve their clients, support their caregivers, and streamline their operations.

Thus, there is a need for case management software that helps to increase case manager productivity, improve quality of care, and allow case managers to serve a larger client pool.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

In some embodiments, a case management platform may comprise one or more hardware processors and one or more non-transitory computer-readable storage media storing instructions. The instructions, when executed by the one or more hardware processors, may cause the one or more hardware processors to perform operations. The operations may comprise connecting to one or more external communication sources and one or more software systems to monitor for notifications. The operations may comprise receiving a notification comprising at least one of a communication from a stakeholder via the one or more communication sources, and an event notification from the one or more software systems. The operations may comprise parsing the notification to determine information relevant to performing one or more actions. The operations may comprise, based on the information contained in the notification, causing one or more actions to be performed. The one or more actions may comprise one or more of executing one or more operations in at least one software system of the one or more software systems, transmitting a synthesized voice communication to one or more stakeholders associated with the notification, and storing records associated with the notification to a database.

In other embodiments, a method for case management may comprise connecting to one or more communication sources and one or more software systems to monitor for notifications. The method may comprise receiving a notification comprising at least one of a communication from a stakeholder via the one or more communication sources, and an event notification from the one or more software systems. The method may comprise parsing the notification to determine information relevant to performing one or more actions. The method may comprise, based on the information contained in the notification, causing one or more actions to be performed. The one or more actions may comprise one or more of executing one or more operations in at least one software system of the one or more software systems, transmitting a synthesized voice communication to one or more stakeholders associated with the notification, and storing records associated with the notification to a database.

In still other embodiments, one or more non-transitory computer-readable storage media may store instructions which, when executed by one or more hardware processors, may cause the one or more hardware processors to perform operations. The operations may comprise connecting to one or more communication sources and one or more software systems to monitor for notifications. The operations may comprise receiving a notification comprising at least one of a communication from a stakeholder via the one or more communication sources, and an event notification from the one or more software systems. The operations may comprise parsing the notification to determine information relevant to performing one or more actions. The operations may comprise, based on the information contained in the notification, causing one or more actions to be performed. The one or more actions may comprise one or more of executing one or more operations in at least one software system of the one or more software systems, transmitting a synthesized voice communication to one or more stakeholders associated with the notification, and storing records associated with the notification to a database. In further aspects, the one or more communication sources may be external communication sources.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates a block diagram of an operating environment consistent with the present disclosure;

FIG. 2 is a flow chart of a method for providing a case management platform;

FIG. 3 is a data flow diagram illustrating an example dataflow for completing a particular task using the case management software; and

FIG. 4 is a block diagram of a system including a computing device for performing the method of FIG. 2.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely to provide a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such a term to mean based on the contextual use of the term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Regarding applicability of 35 U.S.C. § 112, 16, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subject matter disclosed under the header.

The present disclosure includes many aspects and features. Moreover, while many aspects and features relate to, and are described in, the context of case management software (e.g., for a home care agency), embodiments of the present disclosure are not limited to use only in this context. As used herein, the term home care includes home healthcare and any health services that are provided in a patient's home by a caregiver. These services include, but are not limited to, nursing care, dressing assistance. medication assistance, physical therapy, and/or the like.

### I. PLATFORM OVERVIEW

This overview is provided to introduce a selection of concepts in a simplified form that are further described below. This overview is not intended to identify key features or essential features of the claimed subject matter. Nor is this overview intended to be used to limit the claimed subject matter's scope.

In embodiments, a case management platform may make use of a type of artificial intelligence called large language models (LLM) to aid in execution of at least some labor-intensive tasks traditionally carried out by Home Care Agency (HCA) including, but not limited to, a home healthcare agency, employees (e.g., case managers and/or supervisors). The case management tasks the platform may perform include (but are not limited to):
Managing Authorized Hours: Ensuring caregiver staff fulfills hours authorized by the payer.
Managing Timecard Exceptions: A timecard exception occurs if there is a discrepancy between the scheduled caregiver visit schedule to the client and the actual visit information. If a discrepancy exists, an exception is generated. When an exception is generated, the platform can contact the caregiver (e.g., using telephone, text message (SMS), email, etc.) to gather information related to the discrepancy. For example, if the caregiver was scheduled to arrive at 9 am and surpassed a designated buffer time for lateness, the platform may contact the caregiver. In some embodiments (e.g., if the caregiver is unreachable), the platform may schedule an alternate caregiver to provide care to the client.
Advocating for Clients: The platform may help to ensure that clients receive the services they need and are treated with respect, advocating for their needs when necessary. For example, if a caregiver is scheduled to perform a particular task (e.g., bathing) on a daily basis, but the client (or a relative or guardian of the client) discovers that the task is not being performed as scheduled or is being performed poorly, the client may contact the HCA through the platform, which may take action by contacting a caregiver supervisor (e.g., a Director of Field Operations), the caregiver themselves, and/or any other parties who can enforce the performance of scheduled tasks. Any (e.g., all) of these actions may be documented and/or recorded for auditing purposes.
Care Coordination: The platform may help to ensure that care is provided in a coordinated manner. For example, the platform may perform scheduling and communication operations for caregivers and/or home care agency management. For example, the platform may contact a caregiver to adjust a scheduled visit time to visit a client if circumstances change for any reason (e.g., the client has a doctor appointment, the client becomes too ill for the scheduled service, etc.).
Benefits Coordination: The platform may include a benefits database or other data store to retain fringe benefits information, and may provide benefits explanations in plain multiple language, designed to explain the range of benefits and services available to caregivers. For example, the platform may communicate sick time accrual, sick time taken/used, balance currently available to a particular caregiver (e.g., via voice call, visual avatar communication, text-based communication such as SMS message or email, etc.). The platform may approve fringe benefit requests (e.g., time-off requests) if the request meets the agency policy requirements (e.g., enough advanced notice given, ample accruals remaining, etc.); the platform may otherwise disapprove the request and/or transfer the request to a person for further review.
Documentation: The platform may maintain records of client interactions with caregivers.
Tracking Caregiver Work Compliance: The platform may maintain records of federal, state, and/or local agency regulations and/or other licensing of one or more (e.g., each) caregiver to help ensure that care delivery is compliant with existing regulations. For example, the platform may ensure that a caregiver has current certifications, evaluations and has met any other medical compliance requirements.
Crisis Management: The platform may adjust caregiver schedules based on reported emergencies or crises, such as a client's sudden hospitalization.
Staff Supervision: The platform may manage caregiver placement, helping to ensure that the caregivers are properly matched to clients (e.g., based on caregiver work requirements and/or client care plans).

The case management platform may handle (e.g., perform, administer) one or more of these tasks at a consistent and trackable level with human-like friendly interactions 24 hours a day. The platform may perform any relevant tasks based on a received input. In some embodiments the platform may interface with one or more other software programs in use by the home care agency, allowing the home care agency to continue to use their existing software systems. The case management platform may receive and respond to inquiries from various stakeholders (e.g., home care agency employees, caregivers, clients, payers, etc.) involved in home health care verbally (e.g., via telephone, written message such as SMS or email, and/or communication through a specialized application such as a smartphone or tablet computer app) using generative artificial intelligence methods that utilize a Large Language Model. The case management platform may use natural language processing technologies to converse in one or more languages, facilitating smooth communication among stakeholders from diverse cultural and/or national backgrounds. One objective of the case management platform is to help ensure that caregivers deliver quality care, covering all authorized service hours for each client. As a central contact point, the case management platform may streamline communication between stakeholders (e.g., caregivers, clients, client family members or guardians, agency management, payers, and the like). The case management platform may be proactive in covering various case manager duties, including those listed above. The platform may identify one or more gaps in client service and, responsive to identifying a gap, may communicate (e.g., via phone, text, and/or push notifications) with an appropriate stakeholder or manager to resolve the identified gap.

In the dynamic landscape of home care services, the instant platform stands out as an advancement, offering myriad advantages over traditional systems.

The platform excels in optimizing the efficiency of care delivery. In particular, the platform helps to ensure that clients receive their prescribed services promptly and accurately, adhering to the Care Plan (CP) outlined by the client's physician.

The platform has the ability to generate substantial cost savings for Home Care Agencies (HCA). By operating substantially continuously and without the constraints of holidays, vacations, or sick days, the platform provides a financially prudent alternative to increasing staffing. This translates to a more sustainable and cost-effective model for the HCA, allowing them to allocate resources more efficiently.

The platform adheres to the Department of Health's (DOH) Emergency Preparedness protocols, helping to ensure the safety of physically vulnerable clients, especially those residing in disaster-prone areas. For example, the tragic events surrounding Hurricane Katrina in the United States serve as a poignant reminder of the dire consequences of slow response within the home care space. The platform addresses this gap, offering a proactive and life-saving solution.

The platform helps to address the financial disparity within the industry. Caregivers, often the lowest-paid workers despite the demanding nature of their job, stand to benefit from the cost savings generated by Open Coordinator. By redirecting funds that would otherwise be spent on operational constraints, agencies can enhance the compensation and support provided to caregivers, acknowledging and valuing their contribution to the well-being of vulnerable clients. In the context of an aging population where the demand for quality care is escalating, the platform can be a transformative force, helping to elevate the standards of home care services. The efficiency, cost-effectiveness, and lifesaving features of the platform provide help to ensure the well-being of clients, and contribute to a more sustainable and equitable ecosystem for caregivers.

The case management platform addresses the technical problem of inefficient and labor-intensive case management tasks in home care agencies. The platform aims to automate and streamline various aspects of case management using artificial intelligence, specifically large language models (LLMs), to improve productivity and quality of care.

Some examples of how the platform solves this problem across different scenarios include:
1. Managing authorized hours: The platform can automatically track and ensure that caregiver staff fulfills the hours authorized by the payer for each client. This eliminates the need for manual tracking and reduces the risk of exceeding or under-utilizing authorized hours.
2. Handling timecard exceptions: When there is a discrepancy between a caregiver's scheduled visit and actual visit information, the platform can automatically detect this exception. It can then contact the caregiver to gather information about the discrepancy, potentially schedule an alternate caregiver if needed, and document the resolution.
3. Advocating for clients: If a client or their family reports that scheduled tasks are not being performed properly, the platform can automatically escalate the issue to the appropriate supervisors or managers. It can also document all actions taken for auditing purposes.
4. Care coordination: The platform can handle scheduling changes and communications between caregivers, clients, and agency management. For example, if a client has a doctor's appointment that conflicts with a scheduled visit, the platform can automatically adjust the caregiver's schedule and notify all relevant parties.
5. Benefits coordination: The platform can provide caregivers with up-to-date information about their benefits, such as accrued sick time. It can also handle and document requests for time off, approving them automatically if they meet policy requirements or routing them for further review if needed.
6. Crisis management: In emergency situations, such as a client's sudden hospitalization, the platform can quickly adjust caregiver schedules and notify all affected parties.
7. Staff supervision: The platform can manage caregiver placement by matching caregivers to clients based on work requirements and care plans, ensuring appropriate staffing for each client's needs.

One use case focuses on automating these tasks to allow case managers to handle a larger client pool more efficiently. By using natural language processing and machine learning, the platform can interact with stakeholders in a human-like manner, handle routine tasks automatically, and escalate complex issues when necessary. This reduces the manual workload on case managers, improves response times, and ensures consistent handling of tasks across the agency.

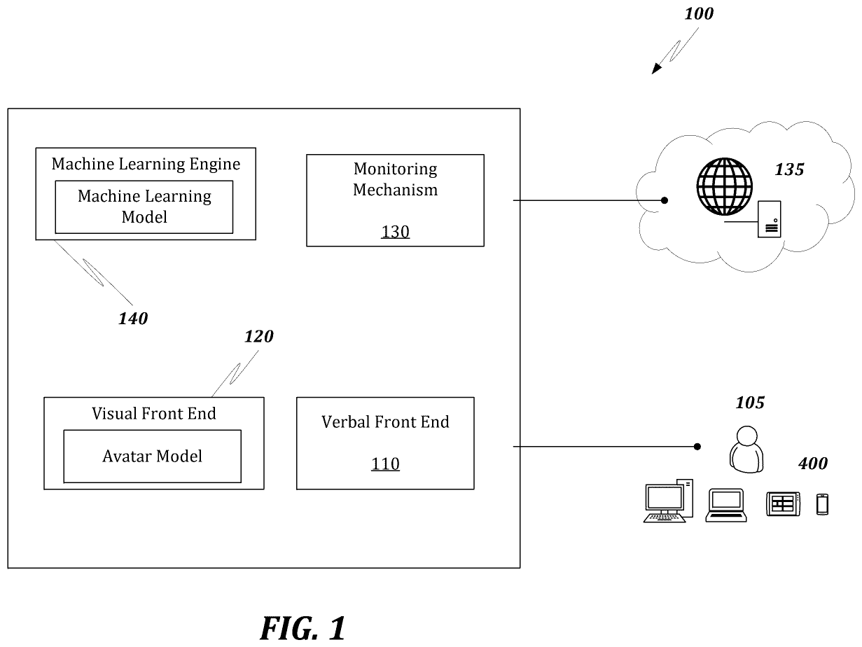

With reference to FIG. 1, the operating environment for enabling embodiments of the present disclosure may include a case management platform 100 that interfaces with various external systems and stakeholders. The platform 100 may be hosted on a cloud computing service or on a dedicated computing device 400.

The platform 100 comprises several key components that work together to provide case management functionality:

A verbal front end 110 may be configured to interpret voice and text communications from stakeholders 105 such as clients, caregivers, agency staff, etc. The verbal front end 110 may utilize natural language processing and speech-to-text capabilities to convert stakeholder communications into a format that can be processed by other components of the platform.

A visual front end 120 may generate a visual avatar interface for interacting with stakeholders. This allows the platform to present information visually and simulate a video call-like experience.

A monitoring mechanism 130 may continuously monitor various software systems 135 used by the home care agency, such as scheduling systems, client management systems, etc. The monitoring mechanism 130 can detect exceptions or events that require action by the platform.

At the core of the platform is a machine learning engine 140 that utilizes one or more machine learning models. This engine processes information from the verbal front end, visual front end, and monitoring mechanism to determine appropriate actions to take.

The platform 100 may interface with various stakeholder devices 105, 400 such as computers, smartphones, tablets etc. to receive communications and provide responses. It may also integrate with existing agency software systems 135 via APIs to access and update relevant data.

This interconnected system allows the platform to serve as a central hub for case management, automating many routine tasks while providing a natural interface for stakeholders to interact with. The cloud-based architecture enables scalability and accessibility from multiple locations and devices.

With reference to FIG. 1, the case management platform 100 may provide a solution for automating various tasks traditionally performed by home care agency employees. The platform 100 may include several key components that work together to provide case management functionality.

A verbal front end 110 may be configured to interpret voice and text communications from stakeholders 105 such as clients, caregivers, agency staff, etc. The verbal front end 110 may utilize natural language processing and speech-to-text capabilities to convert stakeholder communications into a format that can be processed by other components of the platform.

A visual front end 120 may generate a visual avatar interface for interacting with stakeholders. This allows the platform to present information visually and simulate a video call-like experience.

A monitoring mechanism 130 may continuously monitor various software systems 135 used by the home care agency, such as scheduling systems, client management systems, etc. The monitoring mechanism 130 can detect exceptions or events that require action by the platform.

At the core of the platform is a machine learning engine 140 that utilizes one or more machine learning models. This engine processes information from the verbal front end, visual front end, and monitoring mechanism to determine appropriate actions to take.

The platform 100 may interface with various stakeholder devices 105, 400 such as computers, smartphones, tablets etc. to receive communications and provide responses. It may also integrate with existing agency software systems 135 via APIs to access and update relevant data.

This interconnected system allows the platform to serve as a central hub for case management, automating many routine tasks while providing a natural interface for stakeholders to interact with. The cloud-based architecture enables scalability and accessibility from multiple locations and devices.

Some technical advantages of the case management platform over prior art solutions include (but are not limited to):
Automated task handling: The platform can automatically handle many routine case management tasks 24/7 using artificial intelligence, reducing the manual workload on human case managers. This allows case managers to handle a larger client pool more efficiently.
Natural language processing: The verbal front end uses natural language processing to interpret voice and text communications from stakeholders in multiple languages. This enables more natural human-like interactions compared to rigid menu-based systems
Integration with existing systems: The platform can integrate with existing home care agency software systems via APIs, allowing agencies to continue using their current tools while gaining AI assistance. This provides an upgrade path without requiring a complete system overhaul.
Proactive monitoring: The monitoring mechanism continuously monitors agency systems for exceptions or events requiring action, allowing the platform to proactively address issues before they become problems. This improves responsiveness compared to purely reactive systems.
Flexible communication channels: The platform can interact with stakeholders via multiple channels including voice calls, text messages, emails, and custom apps. This provides more flexibility than systems limited to a single communication method.
Visual avatar interface: The visual front end can generate an avatar to visually present information, simulating a video call experience. This enhances engagement compared to voice-only or text-only interfaces.
Machine learning capabilities: The AI module uses machine learning models that can improve over time as they process more data. This allows the system to become more accurate and efficient with use, unlike static rule-based systems.
Scalability: The cloud-based architecture enables easy scaling to handle growing agency needs without requiring hardware upgrades. This provides more flexibility than on-premises solutions.
Comprehensive documentation: The platform automatically documents all interactions and actions taken, creating an audit trail. This improves record-keeping compared to manual documentation processes.
Crisis management: The platform can quickly adjust schedules and notify stakeholders in emergency situations, providing faster response than manual processes.
Multi-stakeholder coordination: By serving as a central hub, the platform can coordinate between multiple stakeholders more efficiently than siloed communication channels.
Compliance tracking: The system can automatically track caregiver certifications and other compliance requirements, reducing the risk of regulatory issues compared to manual tracking.

These technical advantages enable the platform to provide more efficient, responsive, and comprehensive case management compared to traditional manual processes or more limited automation solutions. The AI-driven approach allows for continuous improvement and scalability to meet growing agency needs.

In embodiment a case management platform 100 may include case management software executed by one or more hardware processors. As discussed above, the case management software may perform various functions assistive to the management of cases, either directly or indirectly. The functions performed may include, but need not be limited to, management of authorized caregiving hours for each client, management of timecard exceptions, advocating for clients, care coordination, benefits coordination, documentation, ensuring caregiver work compliance, crisis management, and staff supervision.

In some embodiments, the platform 100 may receive, from a stakeholder, a communication related to case management. The stakeholder may be, as a non-limiting example, a caregiver, a client, a client family member or guardian, an agency manager, a payer, and/or the like. The communication may be, as non-limiting examples, a phone call (e.g., received through a telephone system), a text message, an email, a voice memo and/or text memo submitted through an application, and/or any other data communication between the stakeholder and the platform 100. The platform may use speech-to-text processing and/or natural language processing techniques to obtain an action item from the received communication. Responsive to the action item, the platform 100 may, in some embodiments, engage in a natural language dialogue with the stakeholder to elicit additional information relevant to the communication. Based on the communication and/or the information gathered during the dialogue, the platform 100 may perform one or more actions. As examples, the platform may schedule (and/or modify) one or more caregiver visits and/or initiate one or more communications to one or more stakeholders (e.g., reporting a resolution to the stakeholder who initiated the communication and/or informing additional stakeholders of changes made relating to a client). In embodiments, the platform 100 may create and store documentation relating to the communication, the dialogue, the action item, one or more instructions transmitted to the stakeholder, and/or any other action taken by the platform. The documentation may include conversation transcripts, timestamps, party identifier, and/or any other information useful to the home care agency.

In some embodiments, the platform may include vision artificial intelligence for monitoring and verifying caregiving tasks. The platform may be used to help ensure the execution of caregiving tasks as per a client's care plan. Current methods rely heavily on manual verification or the assumption that the caregiver is making honest claims which can be invasive and inconsistent.

The platform may employ a novel system and method for automated verification of caregiving tasks using vision artificial intelligence (AI) to observe the caregiver in the patient home during the appointed times. This system utilizes a network of cameras installed in a client's home, interfaced with a vision AI. The AI is programmed to interpret the video feed and identify the completion of specific tasks as outlined in the client's care plan.

The camera network may include one or more strategically placed cameras in the client's home, capturing real-time video feeds. A vision AI Module may include an algorithm trained to analyze the video feed and recognize specific caregiving tasks. The algorithm may operate using image recognition and machine learning principles to ensure accurate identification of task execution. A Data Processing Unit may receive and process the output of the vision AI, updating the task completion status in the client's care plan. Security and Privacy Protocols may be used t help ensure that the video feed is exclusively processed by the AI, with human viewing restricted to audit purposes only. Audits are initiated under specific conditions, such as formal requests from the client, advocate, and/or caregiver. A Notification and Reporting System may notify concerned parties (e.g., caregiver supervisors, client guardians, etc.) about task completion, and/or generate periodic reports for review. This system offers enhanced accuracy of the monitoring of caregiving tasks, reduces the need for manual supervision, and maintains privacy and security for the client.

Embodiments of the present disclosure may comprise methods, systems, and a computer readable medium comprising, but not limited to, at least one of the following:
A. A Verbal Front End;
B. A Visual Front End;
C. A Monitoring Mechanism; and
D. An Artificial Intelligence Module.

Details with regard to each module are provided below. Although modules are disclosed with specific functionality, it should be understood that functionality may be shared between modules, with some functions split between modules, while other functions duplicated by the modules. Furthermore, the name of each module should not be construed as limiting upon the functionality of the module. Moreover, each component disclosed within each module can be considered independently, without the context of the other components within the same module or different modules. Each component may contain functionality defined in other portions of this specification. Each component disclosed for one module may be mixed with the functionality of other modules. In the present disclosure, each component can be claimed on its own and/or interchangeably with other components of other modules.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

### II. PLATFORM CONFIGURATION

FIG. 1 illustrates one possible operating environment through which a platform consistent with embodiments of the present disclosure may be provided. By way of non-limiting example, a case management platform 100 may be hosted on, for example, a cloud computing service. In some embodiments, the platform 100 may be hosted on a computing device 400. A user may access platform 100 through a software application and/or hardware device. The software application may be embodied as, for example, but not be limited to, a website, a web application, a desktop application, and a mobile application compatible with the computing device 400.

The platform 100 may integrate with external software systems (e.g., various HCA software systems) using various APIs, data formats, and protocols. The platform may implement a microservices architecture with dedicated integration services for each external system. This allows for flexible, scalable integrations that can evolve independently as external systems change over time.

As described above, the platform 100 may communicate with one or more relevant HCA personnel as required to fulfill agency goals, agency functions, employee functions, etc. The platform may include one or more backend components which poll for events to be addressed by the platform. Accordingly, embodiments of the present disclosure provide a software and hardware platform comprised of a distributed set of computing elements, including, but not limited to:

### A. A Verbal Front End

In embodiments, the platform 100 may include a verbal front end 110. The Verbal front end 110 may include hardware and/or software configured to interpret communications from stakeholders (e.g., clients, advocates, employees, payers, etc.) 105 related to tasks associated with the HCA. The communications may comprise voice communications and/or textual communications. In some embodiments, the interpretation is performed in real time or substantially in real time as the information (e.g., the voice communication) is received.

In particular, the verbal front end 110 may convert a caller's speech to text using one or more natural language processing algorithms. The textual communication may be provided to a machine learning engine, such as a Large Language Model (LLM) or other generative machine learning engine for interpretation.

The large language model (LLM) may be specifically incorporated into and used by the machine learning engine. The machine learning engine may include an LLM component alongside other ML models and algorithms. The LLM may be implemented as a transformer-based neural network architecture, pre-trained on a large corpus of text data. The LLM may interface with other components of the platform via an API layer that handles input/output processing. A fine-tuning module may allow the LLM to be further trained on domain-specific data relevant to case management tasks. An inference engine may help to coordinate passing inputs to the LLM and processing its outputs.

The algorithm for implementing and using the LLM may include, but need not be limited to, the following steps. The platform 100 may Initialize the pre-trained LLM model and load fine-tuned weights for case management domain. The platform 100 may receive input text from other platform components (e.g. parsed communication or exception data). The input data may be preprocessed and/or encoded into a format suitable for the LLM. Thereafter, the preprocessed and/or encoded data may be input through the LLM to generate output text. The LLM output may be processed to extract relevant information, actions, or responses, and the processed output may be passed to one or more other platform components to execute actions. The LLM interaction may be logged for further fine-tuning of the model. Periodically the LLM may be retrained or fine-tuned on new case management data to improve performance.

The LLM may help to enable the machine learning engine to understand natural language inputs, generate human-like responses, and determine appropriate actions based on context. This allows for more flexible and intelligent automation of case management tasks compared to rule-based systems.

The verbal front end 110 may receive, from the machine learning engine, a response to the communication. The response may be provided as a textual output and/or a synthesized speech output. In some embodiments (e.g., where the communication is received as verbal communication and the response is received as a textual response, the verbal front end 110 may utilize a text to speech service to convert the textual response to a synthesized speech response. The verbal front end may use a realistic human-like voice generated through text-to-speech (TTS) services to respond to the communication.

The platform may generate and transmit synthesized voice communications to stakeholders. The platform may receive information about the communication to be sent, including the recipient stakeholder, content, and any relevant context. The natural language processing module analyzes the input to extract key information and intent, and the machine learning engine uses the processed input to generate appropriate response text for the communication.

The text-to-speech module may convert the generated text into synthesized speech audio. An appropriate voice model may be selected, and prosody and/or intonation effects may be applied. Phonemes and acoustic features are generated, and the audio waveform corresponding to the speech is generated. The synthesized speech may be optimized for clarity, naturalness, and transmission.

The platform determines the appropriate channel to deliver the voice communication (e.g. phone call, voicemail, in-app audio message). Thereafter, the communication module initiates the transmission of the synthesized voice message to the recipient stakeholder via the selected delivery method. The platform may track the status of the transmission and confirms successful delivery. Details of the voice communication are logged in the database for record-keeping and future reference. The platform may listen for and process any response from the stakeholder to the voice communication.

This process allows the platform to generate human-like voice communications tailored to each stakeholder interaction in an automated yet natural way. The synthesized voice provides a personal touch while enabling efficient, scalable stakeholder communications.

HCA personnel can initiate a communication to (e.g., call) the platform 100 directly when they require a task to be performed instead of having to login to the HCA software and perform the task manually. The verbal front end 110 and/or the machine learning engine may parse a communication for one or more understand eligible HCA task requests. The verbal front end 110 may integrate with existing HCA software using for example, public, private and/or custom API's. In this way, the verbal front end 110 may transmit information related to a task to one or more HCA software systems to perform one or more actions based on the communication.

As one non-limiting example, an employee transmits a verbal communication (e.g., via a telephone call or mobile app communication) to schedule a sick day. The verbal front end 110 may receive the request and submit the request to an LLM. The LLM may respond with cues that allow the employee to verify eligibility, sick day accruals to date, and other relevant data. The verbal front end may trigger the task in a software system of record with the HCA via one or more API calls. In some embodiments, the system may further provide a verbal response that the sick day has been scheduled. A stakeholder may initiate a communication to the platform 100, handled by the verbal front end 110, to perform any task recognized by the verbal front end and pertinent to their stakeholder type (e.g., client, client advocate, attendant, etc.) and security level.

### B. Visual Front End

In embodiments, the platform 100 may include a visual front end 120. The visual front end 120 may include hardware and/or software configured to provide a visual avatar for the platform 100. The avatar produced by the visual front end 120 may interact with a user (e.g., a stakeholder) in a manner similar to a video call. In some embodiments, the visual front end 120 may display relevant information based on a communication received from the stakeholder. For example, the visual information may include, but need not be limited to, charts, graphs, and/or tables. In some embodiments, the avatar generated by the visual front end may interact with the relevant visual information. For example, the avatar may point to the visual information while the platform 100 shares the information with the user verbally (e.g., using the verbal front end 110). The avatar may be configured to move various body parts (e.g., eyes, mouth, hands, etc.) in realistic human ways during presentation of the information. For example, the avatar mouth may during provision of a response from the verbal front end 110, such that it appears as though the avatar is speaking. As another example, if the user requests to schedule a sick day, the visual front end 120 may display a chart showing accrued sick time for the user, and may cause the avatar to gesture towards the chart when providing the user with an overview of their accrued sick time.

The visual avatar generated by the visual front end 120 may be configured to interact with the stakeholder in a variety of ways beyond simply displaying information. The visual avatar may gesture and/or point to relevant visual information displayed on screen, such as charts, graphs, or documents. For example, the avatar may use hand gestures to highlight specific data points or sections of text as it discusses them verbally. The visual avatar may be configured to have facial expressions and body language that convey emotion and engagement, helping to make the interaction feel more natural and human-like. The avatar may smile, nod, look thoughtful, etc. as appropriate to the conversation. Lip syncing may be used to match the synthesized speech output with avatar movements, creating the illusion that the avatar is actually speaking. The avatar may appear to maintain eye contact and gaze in a direction that follows the user's face/eyes (e.g., if detected by a camera), enhancing the sense of connection. Hand and/or arm movements of the avatar may accompany speech, similar to how a human might gesture while talking.
The avatar may have the ability to move around the screen or zoom in/out as needed to focus on different visual elements. The avatar may be capable of manipulating interactive elements, like buttons, sliders, or other UI components.

In embodiments, the avatar may have a customizable appearance (e.g. clothing, hairstyle, etc.) that can be tailored to the stakeholder's preferences. The avatar may be programmed with animation sequences for common actions like greeting the user, thinking/processing, or saying goodbye. In some embodiments, the view of the avatar may switch between full-body and head/shoulders views as appropriate (e.g., based on what the avatar is asked to do). The behavior of the avatar may respond based on user input, like nodding in acknowledgment when the user speaks.

In embodiment, the avatar may be integrated with other interface elements, allowing the avatar to transition between interacting with the user and interfacing with other parts of the application. The goal of presenting the avatar is to create an engaging, intuitive interface that leverages the avatar's visual presence to enhance communication and usability. The avatar serves as a friendly “face” for the AI system, making interactions feel more personal and approachable for stakeholders.

In some embodiments, the visual front end 120 may provide a textual chat interface, allowing a stakeholder to provide a communication via a text chat using normal human terms in place of or in addition to verbal communication.

### C. Monitoring Mechanism

In embodiments, the platform 100 may include a monitoring mechanism 130. The monitoring mechanism 130 may include hardware and/or software configured to monitor one or more software systems 135 associated with the HCA for changes reflective of tasks to be performed by the platform 100.

In some embodiments, the monitoring mechanism 130 may include a polling mechanism configured to poll one or more software systems associated with the HCA. The polling mechanism may poll at predefined time intervals to monitor the state of the HCA and take appropriate action as necessary to keep the agency in accord. Alternatively or additionally, the monitoring mechanism may be configured to receive updates from software systems (e.g., via an API) when one or more conditions relevant to the HCA change.

Additionally or alternatively, the monitoring mechanism 130 may make one or more API calls to query the status of different systems (e.g., scheduling, client management, caregiver management, payroll, timecards, compliance tracking, etc.) at set frequencies (e.g. every minute) and comparing the current system states against previous states to identify changes. The monitoring mechanism 130 may apply predefined rules to determine if a change constitutes an actionable exception.

In some embodiments, the monitoring mechanism 130 may include an event-driven mechanism where the monitoring mechanism receives real-time notifications from integrated systems when relevant events occur. The monitoring mechanism may establish API connections that allow HCA software systems to push notifications to the platform, and configure systems to generate alerts for specific trigger events (e.g., missed check-ins, approaching authorization deadlines, etc.). The monitoring mechanism may implement message queues to reliably receive and process incoming notifications.

In some embodiments, the monitoring mechanism 130 may include a data streaming mechanism to continuously ingest and analyze relevant data feeds. One or more data pipelines may be set up from operational databases and/or log files. The data streaming mechanism may apply stream processing to detect patterns or anomalies in real-time, and may trigger alerts when predefined thresholds or conditions are met.

The monitoring mechanism 130 may interface with external systems through one or more of RESTful APIs to query data and invoke operations. webhooks to receive push notifications, database connections to directly access relevant tables, file transfers to exchange batch data updates, and/or message queues for asynchronous communication.

This proactive monitoring approach allows the platform to rapidly identify and respond to events requiring action across the HCA's operations.

As a non-limiting example, the platform 100 may determine, via the monitoring mechanism 130, that a home attendant is late, creating a lapse in client service. In some embodiments, the monitoring mechanism may make such a determination based on a lack of check-in from the home attendant within a particular threshold time of an appointment. The system may determine that no check-in has been made based on results of polling a check-in system, and/or because the check-in system has not notified the platform (e.g., via an API call) that a check-in related to the appointment was made. Responsive to the determination, the monitoring mechanism may generate an exception that indicates an issue. Responsive to the exception, the platform 100 may contact (e.g., via a telephone call, text message, in-app voice or text message, etc.) the home attendant who has not yet arrived. The system may determine, based on one or more verbal or textual responses from the home attendant, a status and arrival time for the attendant. If the attendant cannot arrive within a defined time period relative to the appointment, the platform 100 may cancel the appointment on the attendant's schedule and assign a new available home attendant. In embodiments, scheduling a new available home attendant may include calling one or more (e.g., each) attendant who has a status of ‘available’ during the appointment time slot. The calls may be made sequentially (e.g., one by one until an employee indicates an ability to cover the time slot) or simultaneously (e.g., to a plurality of employees at substantially the same time). During the call, the platform 100 may validate, via a human-like conversation (e.g., provided through the verbal front end 110) whether the home attendant can work during the appointment time slot, and when the home attendant can arrive at the appointment location. The platform 100 may update the new home attendant's schedule to include the appointment. The platform 100 (via the monitoring mechanism 130) may resume monitoring for additional exceptions.

### D. Artificial Intelligence Module

In embodiments, the platform 100 may include an Artificial Intelligence (AI) module 140. The AI module 140 may include hardware and/or software configured to process information received from one or more sources and provide an output.

Artificial intelligence may refer to the field of studying artificial intelligence or the methodology for making artificial intelligence. Machine learning refers to the field of defining various issues dealt with in the field of artificial intelligence and studying methodology for solving the various issues. Machine learning may, in some cases, be defined as an algorithm that enhances the performance of a certain task through a steady experience with the certain task.

An artificial neural network (ANN) is a model used in machine learning and may refer to a whole model of problem-solving ability which may be composed of artificial neurons (nodes) that form a network by synaptic connections. The artificial neural network may be defined by a connection pattern between neurons in different layers, a learning process for updating model parameters, and/or an activation function for generating an output value.

The artificial neural network may include an input layer, an output layer, and optionally one or more hidden layers. Each layer includes one or more neurons, and the artificial neural network may include one or more synapses that link one neuron to another neuron. In the artificial neural network, each neuron may output a function value of an activation function for input signals, weights, and deflections input to the neuron.

Model parameters may refer to parameters determined through learning and may include a weight value of synaptic connection and deflection of neurons. A hyperparameter may be a parameter to be set in the machine learning algorithm before learning. In embodiments, a hyperparameter may include a learning rate. a repetition number. a batch size, and/or an initialization function.

The artificial neural network may be used to determine the model parameters that minimize a loss function. The loss function may be used as an index to determine optimal model parameters in the learning process of the artificial neural network. In this way, the artificial neural network may improve its learning capabilities as it receives additional data.

Machine learning may be classified into one or more of supervised learning, unsupervised learning, or reinforcement learning according to a learning method. Supervised learning may refer to a method of learning using an artificial neural network in a state in which a label for learning data is given, and the label may include the correct answer (or result value) that the artificial neural network must infer when the learning data (also known as “training data”) is input to the artificial neural network. That is, lin supervised learning, labeled training data includes input/output pairs in which each input is labeled with a desired output (e.g., a label, classification, and/or categorization), also referred to as a supervisory signal. Unsupervised learning may refer to a method of learning using an artificial neural network in a state in which a label for learning data is not given. That is, in unsupervised learning, the training data does not include supervisory signals. Semi-supervised learning is a hybrid of supervised learning and unsupervised learning. In semi-supervised learning, some inputs are associated with supervisory signals and other inputs are not associated with supervisory signals. Reinforcement learning may refer to a learning method in which an agent defined in a certain environment receives feedback about decisions made using the agent, and the agent learns to select a behavior or a behavior sequence that maximizes cumulative compensation (e.g., positive feedback) in each state. That is, reinforcement learning uses a feedback system in which the machine learning engine receives positive and/or negative reinforcement in the process of attempting to solve a particular problem (e.g., to optimize performance in a particular scenario, according to one or more predefined performance criteria).

In some embodiments, machine learning may be implemented as a deep neural network (DNN) including a plurality of hidden layers of one or more artificial neural networks. Machine learning using such deep neural networks may be referred to as deep learning.

In some embodiments, the machine learning may be implemented as a Large Language Model (LLM). The LLM used in the machine learning engine 140 may be integrated into the overall system architecture. For example, the LLM may be implemented as a cloud-based service that is called via API by the case management platform. This allows the computationally intensive LLM to be hosted separately from the main platform for scalability.

The LLM may be fine-tuned on domain-specific data related to home care agency operations, case management tasks, and relevant regulations. This fine-tuning process may involve collecting a large dataset of historical case management information (e.g., interactions, notes, and/or decisions). The data set may be preprocessed and/or cleaned. Fine-tuning the base LLM on the domain-specific data may include using techniques such as continued pre-training or instruction tuning. The fine-tuned LLM may be utilized for different case management functions such as parsing and understanding incoming communications from stakeholders, generating natural language responses and instructions, extracting key information from unstructured text, classifying issues and determining appropriate actions, summarizing case notes and generating reports, and/or any other tasks for which an LLM is suited.

Some specific examples of LLM utilization may include (but need not be limited to) interpreting a caregiver's text message about a schedule change and determining the appropriate updates to make in the scheduling system, generating a natural language explanation of benefits to a client based on their specific coverage details, extracting key medical information from unstructured clinical notes to populate structured fields, and/or the like.

The base LLM architecture may may include one or more customizations such as: adding domain-specific tokens to the vocabulary, modifying the context window of the LLM to handle longer case management interactions, implementing retrieval-augmented generation to access external knowledge bases, adding control codes to steer the model output for different tasks, and/or other customizations to make the model more suitable for the specific case management tasks for which it is to be used.

The LLM may be tightly integrated with other platform components, including the verbal front-end (for natural conversations) and the monitoring mechanism (to process detected exceptions).

In some embodiments, the AI module 140 may include one or more machine learning engines and one or more machine learning models. A machine learning engine may train a machine learning model to perform one or more operations. Training a machine learning model may use training data to generate a function that, given one or more inputs to the machine learning model, computes a corresponding output. The output may correspond to a prediction based on prior machine learning. In an embodiment, the output includes a label, classification, and/or categorization assigned to the provided input(s). The machine learning model corresponds to a learned model for performing the desired operation(s) (e.g., labeling, classifying, and/or categorizing inputs). The AI module 140 may use multiple machine learning engines and/or multiple machine learning models for different purposes.

In an embodiment, the AI module 140 may use supervised learning, semi-supervised learning, unsupervised learning, reinforcement learning, and/or any other training method or combination thereof. In an embodiment, the machine learning engine may initially use supervised learning to train the machine learning model, and then may use unsupervised learning to update the machine learning model on an ongoing basis.

The platform may utilize one or more types of machine learning models, including (but not limited to): LLMs, classification models, sequence-to-sequence models, reinforcement learning models, and/or any other machine learning model type useful in the processes performed by the platform. LLMs may be used for natural language processing tasks like parsing communications, generating responses, and/or determining appropriate actions. As specific, non-limiting examples, the platform may use LLMs such as GPT-3, GPT-4, and/or open source alternatives like BLOOM or LLAMA.

Classification models may be used for tasks such as categorizing notifications, determining urgency, and/or routing to appropriate systems/stakeholders. The classification models may be implemented as neural networks or simpler models like random forests.

Sequence-to-sequence models may be used for tasks like speech-to-text conversion of voice communications. The sequence-to-sequence models may be based on transformer architectures.

Reinforcement learning models may be used to help optimize or improve decision making over time based on feedback and outcomes.

The models are trained through a multi-stage process. In an initial pre-training stage, the system may ingest large corpora of text data to develop general language understanding capabilities. The model may then undergo a fine-tuning on domain-specific datasets related to healthcare, case management, and/or relevant regulations/policies. This data may be curated and anonymized, and may be based on historical case records. The model may continue learning through federated learning approaches, allowing the models to improve based on real-world usage while preserving privacy. Regular retraining and updating of models may be used to incorporate new data and/or adjust for concept drift over time.

The models make predictions and decisions through the following process. Communications or notifications are processed (e.g., parsed) to extract relevant features. The extracted features may be converted to an appropriate input format for the models. One or more models may be queried using the extracted data as an input. In some embodiments, multiple models may be queried in parallel to analyze different aspects of the input (e.g. intent classification, entity extraction, sentiment analysis). Results (e.g., output) of the model(s) may be combined with relevant context from knowledge bases and historical data. Based on the aggregated analysis, the system may determine one or more appropriate actions using decision trees and/or other logic. Each potential action may be assigned a confidence score. Actions below a certain threshold may be flagged for human review. One or more selected actions may be carried out, potentially involving further model inference for tasks like natural language generation.

In an embodiment, the AI module 140 may use many different techniques to label, classify, and/or categorize inputs. A machine learning engine may transform inputs into feature vectors that describe one or more properties (“features”) of the inputs. The machine learning engine may label, classify, and/or categorize the inputs based on the feature vectors. Alternatively or additionally, a machine learning engine may use clustering (also referred to as cluster analysis) to identify commonalities in the inputs. The machine learning engine may group (i.e., cluster) the inputs based on those commonalities. The machine learning engine may use hierarchical clustering, k-means clustering, and/or another clustering method or combination thereof. In an embodiment, a machine learning engine may include an artificial neural network. As discussed above, an artificial neural network includes multiple nodes (also referred to as artificial neurons) and edges (synapses) between the nodes. Edges may be associated with corresponding weights that represent the strengths of connections between nodes, which the machine learning engine may adjust as machine learning proceeds. Alternatively or additionally, a machine learning engine may include a support vector machine. A support vector machine represents inputs as vectors. The machine learning engine may label, classify, and/or categorize inputs based on the vectors. Alternatively or additionally, the machine learning engine may use a naïve Bayes classifier to label, classify, and/or categorize inputs. Alternatively or additionally, given a particular input, a machine learning model may apply a decision tree to predict an output for the given input. Alternatively or additionally, a machine learning engine may apply fuzzy logic in situations where labeling, classifying, and/or categorizing an input among a fixed set of mutually exclusive options is impossible or impractical. The aforementioned machine learning model and techniques are discussed for exemplary purposes only and should not be construed as limiting one or more embodiments.

For example, the AI module 140 may receive, as inputs, information from a stakeholder (e.g., collected by the verbal front end) and/or information regarding an exception (e.g., detected by the monitoring mechanism 130). The machine learning engine may associate one or more actions (to correct the issues noted int eh communication and/or exception) with the input information. Alternatively or additionally, the machine learning engine may associate one or more information requests with the input information. In embodiments, the one or more actions and/or information requests associated with the information may be selected from defined sets of actions and/or information requests.

In one embodiment, as a machine learning engine applies different inputs to a machine learning model, the corresponding outputs are not always accurate. As an example, the machine learning engine may use supervised learning to train a machine learning model. After training the machine learning model, if a subsequent input is identical to an input that was included in labeled training data and the output is identical to the supervisory signal in the training data, then output is certain to be accurate. If an input is different from inputs that were included in labeled training data, then the machine learning engine may generate a corresponding output that is inaccurate or of uncertain accuracy. In addition to producing a particular output for a given input, the machine learning engine may be configured to produce an indicator representing a confidence (or lack thereof) in the accuracy of the output. A confidence indicator may include a numeric score, a Boolean value, and/or any other kind of indicator that corresponds to a confidence (or lack thereof) in the accuracy of the output.

### III. PLATFORM OPERATION

Embodiments of the present disclosure provide a hardware and software platform operative by a set of methods and computer-readable media comprising instructions configured to operate the aforementioned modules and computing elements in accordance with the methods. The following depicts an example of at least one method of a plurality of methods that may be performed by at least one of the aforementioned modules. Various hardware components may be used at the various stages of operations disclosed with reference to each module.

For example, although methods may be described as being performed by a single computing device, it should be understood that, in some embodiments, different operations may be performed by different networked elements in operative communication with the computing device. For example, at least one computing device 400 may be employed in the performance of some or all of the stages disclosed with regard to the methods. Similarly, an apparatus may be employed in the performance of some or all of the stages of the methods. As such, the apparatus may comprise at least those architectural components found in computing device 400.

Furthermore, although the stages of the following example method are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones described below. Moreover, various stages may be added or removed from the without altering or departing from the fundamental scope of the depicted methods and systems disclosed herein.

### A. Master Method

Consistent with embodiments of the present disclosure, a method may be performed by at least one of the aforementioned modules. The method may be embodied as, for example, but not limited to, computer instructions, which, when executed, perform the method.

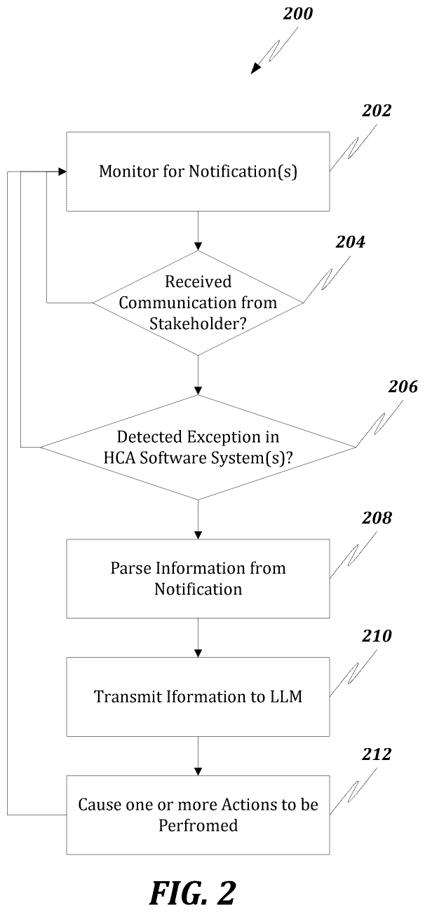

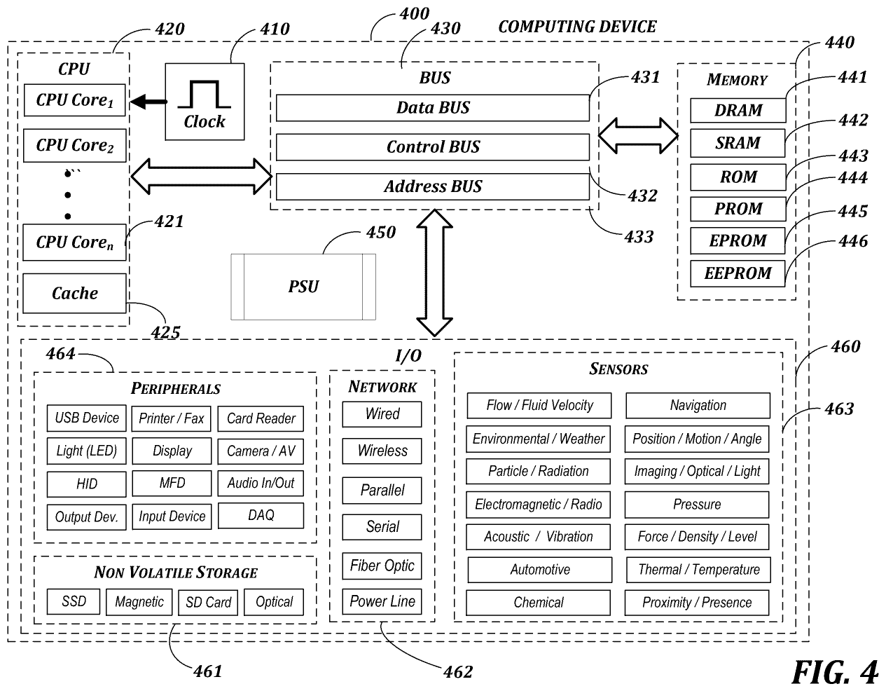

FIG. 2 is a flow chart setting forth the general stages involved in a method 200 consistent with an embodiment of the disclosure for providing a case management platform 100. Method 200 may be implemented using a computing device 400 or any other component associated with platform 100 as described in more detail below with respect to FIG. 4. For illustrative purposes alone, computing device 400 is described as one potential actor in the following stages.

Method 200 may begin at starting block 205 and proceed to stage 210 where computing device 400 may monitor the HCA for tasks to be performed. For example, the computing device may monitor for one or more communications received from a platform stakeholder (e.g., a caregiver, a client, a client family member or guardian, an agency manager, a payer, and/or the like) and/or one or more exceptions generated by systems used at the HCA to track HCA operations.

At stage 215, the computing device may determine whether a communication has been received. The communication may include, for example, a voice communication (e.g., a telephone call, a voice memo, etc.), a text communication (e.g., a text message, an email, an electronic message delivered through a proprietary application, etc.), and/or any other communication that includes information relevant to allow the platform to perform a task for the HCA.

At stage 220, the computing device may determine whether an exception has been detected. The computing device 400 may monitor the HCA for one or more exceptions using a monitoring mechanism. The one or more exceptions may be determined based on exceptions or errors noted in one or more existing HCA systems. Examples of HCA systems include existing software systems used by the HCA in tracking stakeholder activity. For example, software systems tracking caregiver attendance, client care hours available, payer details, and/or any other aspects of HCA operations tracked by software systems. In some embodiments, the monitoring may be an active monitoring, whereby the computing device may poll the various HCA systems to determine whether any exceptions have been discovered. For example, the computing device may poll each system once per second, once per minute, or at any interval deemed appropriate for the particular system. The computing device may poll each system at the same interval, or may set different intervals for one or more (e.g., each) of the systems. In some embodiments, the monitoring may be a passive monitoring, whereby the computing device is operatively connected to each of the systems using one or more APIs, and each system is configured to transmit a notification to the computing device via the one or more APIs in response to an exception detected at that system.

If the computing device determines in stage 215 that no communication is received and in stage 220 that no exception is detected, the system may return to stage 210, where the computing device may continue to monitor the HCA.

If the computing device receives a communication in stage 215 or detects an exception at stage 220, the method 200 may proceed to stage 225, where the computing device may parse the communication or exception for information. In some embodiments (e.g., where a voice communication is received, parsing the communication may include performing speech to text processing to convert the spoken communication to a text document. The parsing process may extract relevant information from notifications may include the following steps:

For voice communications, speech-to-text processing may first be applied to convert the audio to text. The computing device may use natural language processing techniques on the text document (e.g., the text document generated by speech to text processing, a text communication received directly from the stakeholder, and/or a text report received via the monitoring mechanism). For example, the natural language processing may be used to extract information from the text document. NLP techniques may convert the notification text or speech into a structured format. This may involve tokenization to break the text into individual words or tokens, part-of-speech tagging to identify nouns, verbs, adjectives, etc., named entity recognition to extract key entities like names, dates, locations, and/or dependency parsing to understand grammatical structure and relationships between words.

As an example, information extracted from a communication may include (but need not be limited to) a stakeholder identifier associated with the document origin (e.g., a caller), a subject stakeholder, an action to be taken by the system, metadata including a time of the call and/or a location of the caller, and/or any other information that can be extracted from the communication. Similarly, information extracted from a detected exception may include (but is not limited to) a system that detected the exception, a subject stakeholder, an action to be taken by the system, a time of the exception, and/or any other information that can be extracted from the communication.

The structured text may then be analyzed using machine learning algorithms to identify and extract key information. As examples, the type of notification (e.g. communication from stakeholder, system exception), the stakeholder involved (e.g. caregiver, client, manager), the subject or topic of the notification, any actions or requests mentioned, and/or relevant dates, times, or locations may be identified and extracted.

Domain-specific rules and knowledge bases may be utilized to interpret industry-specific terminology and concepts related to home care agency operations. The extracted information may be mapped to predefined categories and fields to structure it for further processing by the system.

In some embodiments, one or more confidence scores may be assigned to extracted information to indicate the certainty of the extraction. Machine learning models (e.g., neural networks) may be used to classify the overall intent or purpose of the notification. The parsed and extracted information may be formatted into a standardized data structure or JSON object for integration with other system components. Any ambiguous or low-confidence extractions may be flagged for human review. The parsing results may be logged for auditing and to continuously improve the extraction algorithms over time.

This parsing process allows the system to convert unstructured notification data into actionable structured information to drive automated workflows and responses.

The method 200 may proceed to stage 230, where the computing device 400 may transmit the parsed information to a machine learning engine. As examples, the information may be transmitted as a data vector, as a text file, or any other way that the machine learning engine may make use of the data.

In some embodiments, not all information necessary to determine an action to be performed (and/or to perform that action) is contained in the information transmitted to the machine learning model. The system may determine that additional information is needed to perform one or more actions in several ways. As non-limiting examples:
1. Insufficient data: The machine learning model may analyze the parsed information from the notification and determine that key details are missing to execute a specific action. For example, if scheduling a caregiver visit, the system may need the client name, date, time, and service type.
2. Ambiguous request: If the stakeholder's communication is unclear or could be interpreted in multiple ways, the system may need clarification before proceeding.
3. Conflicting information: The system may detect inconsistencies between the new information and existing data in the HCA software systems, requiring verification.
4. Policy compliance: Certain actions may require additional approvals or documentation to comply with HCA policies or regulations.
5. Contextual factors: The system may identify situational factors that necessitate more information, such as changes in a client's condition or caregiver availability.

In such cases, the machine learning model may optionally transmit, to the computing device, a request for additional information. The request for additional information may comprise one or more synthesized vocal requests, one or more written requests, one or more API calls to request information from a software system. In some embodiments. the computing device may communicate the request for more information to a stakeholder. For example, the computing device may initiate a telephone call to a stakeholder, transmit a text or email communication to a stakeholder, transmit a push notification comprising a voice memo and/or written request to a stakeholder via a proprietary application, etc. As a further example, the computing device may transmit one or more instructions to a software system associated with the HCA that may cause the software system to provide additional information to the computing device.

When additional information is needed, the system may generate and transmit a request for the additional information through the following steps:
1. Identify information gap: The machine learning model pinpoints the specific data needed to complete the action.
2. Formulate request: The system generates a clear, concise request for the required information, using natural language processing to craft an appropriate message.
3. Determine recipient: The system identifies the most suitable stakeholder to provide the information, based on the nature of the request and stakeholder roles.
4. Select communication channel: The system chooses the optimal method to transmit the request (e.g., voice call, text message, email, or in-app notification) based on stakeholder preferences and urgency.
5. Generate and send request: The system creates the message in the appropriate format and transmits it through the selected channel. For voice calls, it may use text-to-speech technology to deliver a synthesized voice message.
6. Set follow-up protocol: The system establishes a timeframe for expected response and schedules reminders or escalation procedures if no response is received.
7. Process response: When the additional information is received, the system parses and integrates it with the original data to proceed with the intended action.
8. Update records: The system documents the request for additional information and the response received in the appropriate database for future reference and auditing purposes.

By implementing this process, the case management platform ensures it has complete and accurate information to perform actions effectively while maintaining clear communication with stakeholders.

Responsive to the request for additional information, the computing device may receive and parse the additional information. In some embodiments, the additional information may be received and transmitted in the same way that the initial communication was received and transmitted. Alternatively, the additional information may be transmitted in a different way. For example, the initial communication may have been received via a voice message, as a phone call. The additional information may be received via a text message. In some embodiments, the stakeholder contacted for additional information may be the stakeholder that provided the initial communication. In other situations, the stakeholder contacted for additional information may be the subject of the communication, or another stakeholder, other than the stakeholder who provided the communication (e.g., a caretaker supervisor, a client family member or guardian, etc.).

Once the machine learning engine has received the necessary information, the method 200 may proceed to stage 240, where the machine learning engine may utilize a machine learning model to determine one or more tasks to be performed in response to the received information. In embodiments, the one or more tasks may include providing information to the stakeholder that initiated the communication, providing information to a stakeholder that is the subject of the communication, providing information to one or more additional stakeholders, scheduling one or more appointments, updating one or more calendars, storing data related to the interaction, etc. In some embodiments, the computing device may also provide, to the stakeholder initiating the communication, an indication of the one or more tasks to be carried out by the platform.

In particular, the machine learning engine may provide instructions that, when executed, cause one or more actions to be performed. The instructions may be transmitted to the computing device and/or provided to one or more additional systems associated with the HCA.

The platform may implement actions like advocating for a client, coordinating care, managing a crisis, etc. through various algorithms and steps. For advocating for a client, the platform may receive a notification of a potential issue (e.g. scheduled task not being performed), parse notification to extract key details (client, caregiver, task, frequency, etc.), query a database to retrieve client care plan and history, analyze discrepancies between the care plan and the reported issue, and generate advocacy action plan. Thereafter, the platform may contact caregiver for explanation, escalate to supervisor (if needed), schedule follow-up check with client, and/or document issue and resolution. The platform may execute advocacy actions such as (but not limited to) using natural language processing to conduct conversations, updating relevant systems with new information, and/or creating documentation of advocacy efforts. The platform may monitor for resolution, and may escalate the issue if it remains unresolved after defined period of time.

For coordinating care, the platform may receive care coordination request or detect need from monitoring, retrieve client care plan, caregiver schedules, and other relevant data, and identify care gaps or conflicts. The platform may generate care coordination options. Including (but not limited to) adjusting one or more caregiver schedules, adding/removing caregivers. And/or modifying care tasks or frequency. The platform may evaluate options using defined criteria (e.g. cost, caregiver preferences, etc.), and may select and implement optimal care coordination plan. The platform may update schedules in the system, notify affected caregivers and/or clients of the schedule change, document changes to care plan, and monitor execution of new care plan to adjust as needed.

For managing a crisis, the platform may receive crisis notification assess the severity and type of crisis, and retrieve crisis management protocols. The platform may identify stakeholders to be notified (e.g. emergency services, family, etc.) and execute one or more immediate response actions, such as (but not limited to) contacting emergency services (if needed), notifying designated emergency contacts, and/or dispatching an on-call caregiver. The platform may coordinate ongoing crisis management by providing updates to stakeholders, adjusting care schedules as needed, and/or documenting the crisis and/or the response. The platform may conduct a post-crisis review and update protocols if needed

The platform may use machine learning models to optimize these processes over time based on outcomes and feedback. Natural language processing allows for human-like interactions throughout execution of these algorithms.

After causing execution of the one or more actions, the method 200 may return to stage 210, where the computing device monitors the HCA.

### B. Example Data Flow—Upcoming Service Authorization Expiration

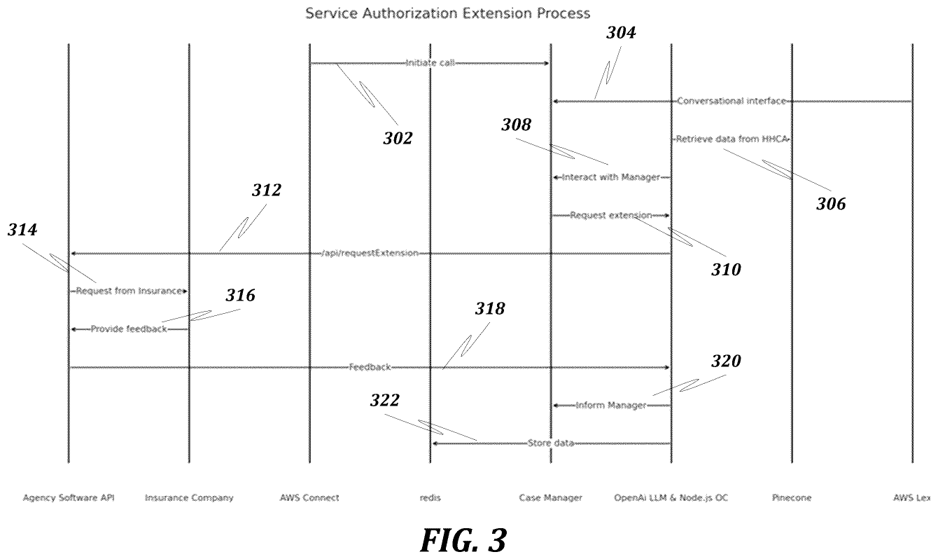

FIG. 3 shows an example data flow illustrating the performance of various aspects of a data flow for addressing an upcoming service authorization end date consistent with an embodiment of the disclosure for providing a case management platform 100.

The HCA may have contracts with different payers (e.g., insurance companies, Medicaid, Medicare, etc.) to service clients with home health care service workers. Each client may be associated with an individual contract (e.g., a service authorization) which stipulates at least a start and an end date, and a number of hours of service eligible as billable to the payer. The service authorization end date is very important to the HCA as, after the end date, the HCA can no longer bill hours for visits to the particular client. This example data flow 300 shows how the platform 100 handles the event of a service authorization end date approaching.

The system may monitor for various events, including an approaching service authorization end date. For example, the system may poll one or more HCA system at regular intervals (e.g., every minute) via a simple code scheduling mechanism. When the approaching service authorization end date event is found (e.g., when the service authorization end date is within a threshold time of the current time), the following happens:

In stage 302, the platform determines one or more recipients. The platform initiates an outgoing call to the determined recipients. For example, the verbal front end may include a contact center software (e.g., AWS Connect, Five9, Genesys, etc.) used to initiate the call. In this example, the determined recipient may be a case manager associated with the client.

In stage 304, the verbal front end may employ a conversational interface service (e.g., AWS Lex, Google Cloud Dialogflow, Azure Bot Services, etc.) to provide a conversational interface. The conversational interface may facilitate interaction with the case manager, so the case manager can verbally instruct the platform on how to handle the upcoming deadline.

In stage 306, a machine learning engine (e.g., a Large Language model generative AI, such as OpenAI, Llama, Cohere, etc.) may retrieve relevant data from the HCA datasets to be used during the conversation. If the case manager asks for information that is not readily available to the LLM or machine learning engine via the existing prompt or context, the LLM may retrieve relevant data from the vector database (e.g., Pinecode, Milvus, Qdrant, etc.) to facilitate a helpful conversation with the case manager.

In stage 308, the LLM may be used to engage in an interactive conversation with the case manager.

In stage 310, the case manager, during the conversation, verbally requests a service authorization extension.

In stage 312, the LLM may process the case manager's request. Responsive to the case manager requesting an extension of the service authorization, the LLM may cause the platform to make an API call to the Agency Software API.

In stage 314, the Agency Software API call may cause the Agency Software to execute one or more instructions to communicate the extension request to the insurance company (e.g., via an API or other data connection between the Agency Software and software operated by the insurance company).

In stage 316, the Agency Software may receive, from the insurance company, The insurance company provides feedback regarding the extension request to the Agency Software. For example, the insurance agency software may communicate the feedback using an API.

In stage 320, the feedback from the insurance company may be relayed to the LLM. For example, an API connecting the Agency Software to the LLM may be used to provide the feedback from the insurance company.

In stage 322, the LLM and the verbal front end may be used to inform the case manager about the feedback received from the insurance company regarding the extension request.

In stage 324, at least a portion (e.g., all) of the conversational data and interactions may be stored in a memory database or data stores (e.g., Redis, memcache) for memory-based and/or persistent storage. Storing the conversational data helps to ensure that data is available for future interactions. In embodiments, the stored data is also useful for audit purposes.

### IV. HARDWARE CONFIGURATION

Embodiments of the present disclosure provide a hardware and software platform operative as a distributed system of modules and computing elements.

Platform 100 may be embodied as, for example, but not be limited to, a website, a web application, a desktop application, a backend application, and a mobile application compatible with a computing device 400. The computing device 400 may comprise, but not be limited to, the following:

Mobile computing device, such as, but is not limited to, a laptop, a tablet, a smartphone, a drone, a wearable, an embedded device, a handheld device, an Arduino, an industrial device, or a remotely operable recording device;

A supercomputer, an exascale supercomputer, a mainframe, or a quantum computer;

A minicomputer, wherein the minicomputer computing device comprises, but is not limited to, an IBM AS400/iSeries/System I, A DEC VAX/PDP, an HP3000, a Honeywell-Bull DPS, a Texas Instruments TI-990, or a Wang Laboratories VS Series;

A microcomputer, wherein the microcomputer computing device comprises, but is not limited to, a server, wherein a server may be rack-mounted, a workstation, an industrial device, a raspberry pi, a desktop, or an embedded device;

Platform 100 may be hosted on a centralized server or a cloud computing service. Although method 200 has been described to be performed by a computing device 400, it should be understood that, in some embodiments, different operations may be performed by a plurality of the computing devices 400 in operative communication on at least one network.

Embodiments of the present disclosure may comprise a system having a central processing unit (CPU) 420, a bus 430, a memory unit 440, a power supply unit (PSU) 450, and one or more Input/Output (I/O) units. The CPU 420 coupled to the memory unit 440 and the plurality of I/O units 460 via the bus 430, all of which are powered by the PSU 450. It should be understood that, in some embodiments, each disclosed unit may actually be a plurality of such units for redundancy, high availability, and/or performance purposes. The combination of the presently disclosed units is configured to perform the stages of any method disclosed herein.

FIG. 4 is a block diagram of a system including computing device 400. Consistent with an embodiment of the disclosure, the aforementioned CPU 420, the bus 430, the memory unit 440, a PSU 450, and the plurality of I/O units 460 may be implemented in a computing device, such as computing device 400 of FIG. 4. Any suitable combination of hardware, software, or firmware may be used to implement the aforementioned units. For example, the CPU 420, the bus 430, and the memory unit 440 may be implemented with computing device 400 or any of other computing devices 400, in combination with computing device 400. The aforementioned system, device, and components are examples and other systems, devices, and components may comprise the aforementioned CPU 420, the bus 430, and the memory unit 440, consistent with embodiments of the disclosure.

At least one computing device 400 may be embodied as any of the computing elements illustrated in all of the attached figures. A computing device 400 does not need to be electronic, nor even have a CPU 420, nor bus 430, nor memory unit 440. The definition of the computing device 400 to a person having ordinary skill in the art is “A device that computes, especially a programmable [usually] electronic machine that performs high-speed mathematical or logical operations or that assembles, stores, correlates, or otherwise processes information.” Any device which processes information qualifies as a computing device 400, especially if the processing is purposeful.

With reference to FIG. 4, a system consistent with an embodiment of the disclosure may include a computing device, such as computing device 400. In some configurations, the computing device 400 may include at least one clock module 410, at least one CPU 420, at least one bus 430, and at least one memory unit 440, at least one PSU 450, and at least one I/O module 460, wherein I/O module may be comprised of, but not limited to a non-volatile storage sub-module 461, a communication sub-module 462, a sensors sub-module 463, and a peripherals sub-module 464.

In a system consistent with an embodiment of the disclosure, the computing device 400 may include the clock module 410, known to a person having ordinary skill in the art as a clock generator, which produces clock signals. Clock signals may oscillate between a high state and a low state at a controllable rate, and may be used to synchronize or coordinate actions of digital circuits. Most integrated circuits (ICs) of sufficient complexity use a clock signal in order to synchronize different parts of the circuit, cycling at a rate slower than the worst-case internal propagation delays. One well-known example of the aforementioned integrated circuit is the CPU 420, the central component of modern computers, which relies on a clock signal. The clock 410 can comprise a plurality of embodiments, such as, but not limited to, a single-phase clock which transmits all clock signals on effectively 1 wire, a two-phase clock which distributes clock signals on two wires, each with non-overlapping pulses, and a four-phase clock which distributes clock signals on 4 wires.

Many computing devices 400 may use a “clock multiplier” which multiplies a lower frequency external clock to the appropriate clock rate of the CPU 420. This allows the CPU 420 to operate at a much higher frequency than the rest of the computing device 400, which affords performance gains in situations where the CPU 420 does not need to wait on an external factor (like memory 440 or input/output 460). Some embodiments of the clock 410 may include dynamic frequency change, where, the time between clock edges can vary widely from one edge to the next and back again.

In a system consistent with an embodiment of the disclosure, the computing device 400 may include the CPU 420 comprising at least one CPU Core 421. In other embodiments, the CPU 420 may include a plurality of identical CPU cores 421, such as, but not limited to, homogeneous multi-core systems. It is also possible for the plurality of CPU cores 421 to comprise different CPU cores 421, such as, but not limited to, heterogeneous multi-core systems, big.LITTLE systems and some AMD accelerated processing units (APU). The CPU 420 reads and executes program instructions which may be used across many application domains, for example, but not limited to, general purpose computing, embedded computing, network computing, digital signal processing (DSP), and graphics processing (GPU). The CPU 420 may run multiple instructions on separate CPU cores 421 simultaneously. The CPU 420 may be integrated into at least one of a single integrated circuit die, and multiple dies in a single chip package. The single integrated circuit die and/or the multiple dies in a single chip package may contain a plurality of other elements of the computing device 400, for example, but not limited to, the clock 410, the bus 430, the memory 440, and I/O 460.

The CPU 420 may contain cache 422 such as but not limited to a level 1 cache, a level 2 cache, a level 3 cache, or combinations thereof. The cache 422 may or may not be shared amongst a plurality of CPU cores 421. The cache 422 sharing may comprise at least one of message passing and inter-core communication methods used for the at least one CPU Core 421 to communicate with the cache 422. The inter-core communication methods may comprise, but not be limited to, bus, ring, two-dimensional mesh, and crossbar. The aforementioned CPU 420 may employ symmetric multiprocessing (SMP) design.

The one or more CPU cores 421 may comprise soft microprocessor cores on a single field programmable gate array (FPGA), such as semiconductor intellectual property cores (IP Core). The architectures of the one or more CPU cores 421 may be based on at least one of, but not limited to, Complex Instruction Set Computing (CISC), Zero Instruction Set Computing (ZISC), and Reduced Instruction Set Computing (RISC). At least one performance-enhancing method may be employed by one or more of the CPU cores 421, for example, but not limited to Instruction-level parallelism (ILP) such as, but not limited to, superscalar pipelining, and Thread-level parallelism (TLP).

Consistent with the embodiments of the present disclosure, the aforementioned computing device 400 may employ a communication system that transfers data between components inside the computing device 400, and/or the plurality of computing devices 400. The aforementioned communication system will be known to a person having ordinary skill in the art as a bus 430. The bus 430 may embody internal and/or external hardware and software components, for example, but not limited to a wire, an optical fiber, various communication protocols, and/or any physical arrangement that provides the same logical function as a parallel electrical bus. The bus 430 may comprise at least one of a parallel bus, wherein the parallel bus carries data words in parallel on multiple wires; and a serial bus, wherein the serial bus carries data in bit-wise serial form. The bus 430 may embody a plurality of topologies, for example, but not limited to, a multidrop/electrical parallel topology, a daisy chain topology, and connected by switched hubs, such as a USB bus. The bus 430 may comprise a plurality of embodiments, for example, but not limited to:
Internal data bus (data bus) 431/Memory bus
Control bus 432
Address bus 433
System Management Bus (SMBus)
Front-Side-Bus (FSB)
External Bus Interface (EBI)
Local bus
Expansion bus
Lightning bus
Controller Area Network (CAN bus)
Camera Link
ExpressCard

Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.
Small Computer System Interface (SCSI)/Serial Attached SCSI (SAS)
HyperTransport
InfiniBand
RapidIO
Mobile Industry Processor Interface (MIPI)
Coherent Processor Interface (CAPI)
Plug-n-play
1-Wire
Peripheral Component Interconnect (PCI), including embodiments such as but not limited to, Accelerated Graphics Port (AGP), Peripheral Component Interconnect extended (PCI-X), Peripheral Component Interconnect Express (PCI-e) (e.g., PCI Express Mini Card, PCI Express M.2 [Mini PCle v2], PCI Express External Cabling [ePCle], and PCI Express OCuLink [Optical Copper {Cu} Link]), Express Card, AdvancedTCA, AMC, Universal 10, Thunderbolt/Mini DisplayPort, Mobile PCle (M-PCle), U.2, and Non-Volatile Memory Express (NVMe)/Non-Volatile Memory Host Controller Interface Specification (NVMHCIS).
Industry Standard Architecture (ISA), including embodiments such as, but not limited to Extended ISA (EISA), PC/XT-bus/PC/AT-bus/PC/104 bus (e.g., PC/104-Plus, PCI/104-Express, PCI/104, and PCI-104), and Low Pin Count (LPC).
Music Instrument Digital Interface (MIDI)
Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1394 Interface/Firewire, Thunderbolt, and extensible Host Controller Interface (xHCI).

Consistent with the embodiments of the present disclosure, the aforementioned computing device 400 may employ hardware integrated circuits that store information for immediate use in the computing device 400, known to persons having ordinary skill in the art as primary storage or memory 440. The memory 440 operates at high speed, distinguishing it from the non-volatile storage sub-module 461, which may be referred to as secondary or tertiary storage, which provides relatively slower access to information but offers higher storage capacity. The data contained in memory 440 may be transferred to secondary storage via techniques such as, but not limited to, virtual memory and swap. The memory 440 may be associated with addressable semiconductor memory, such as integrated circuits consisting of silicon-based transistors, that may be used as primary storage or for other purposes in the computing device 400. The memory 440 may comprise a plurality of embodiments, such as, but not limited to volatile memory, non-volatile memory, and semi-volatile memory. It should be understood by a person having ordinary skill in the art that the following are non-limiting examples of the aforementioned memory:
Volatile memory, which requires power to maintain stored information, for example, but not limited to, Dynamic Random-Access Memory (DRAM) 441, Static Random-Access Memory (SRAM) 442, CPU Cache memory 425, Advanced Random-Access Memory (A-RAM), and other types of primary storage such as Random-Access Memory (RAM).
Non-volatile memory, which can retain stored information even after power is removed, for example, but not limited to, Read-Only Memory (ROM) 443, Programmable ROM (PROM) 444, Erasable PROM (EPROM) 445, Electrically Erasable PROM (EEPROM) 446 (e.g., flash memory and Electrically Alterable PROM [EAPROM]), Mask ROM (MROM), One Time Programmable (OTP) ROM/Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D XPoint, Domain-Wall Memory (DWM), and millipede memory.
Semi-volatile memory may have limited non-volatile duration after power is removed but may lose data after said duration has passed. Semi-volatile memory provides high performance, durability, and other valuable characteristics typically associated with volatile memory, while providing some benefits of true non-volatile memory. The semi-volatile memory may comprise volatile and non-volatile memory, and/or volatile memory with a battery to provide power after power is removed. The semi-volatile memory may comprise, but is not limited to, spin-transfer torque RAM (STT-RAM).

Consistent with the embodiments of the present disclosure, the aforementioned

computing device 400 may employ a communication system between an information processing system, such as the computing device 400, and the outside world, for example, but not limited to, human, environment, and another computing device 400. The aforementioned communication system may be known to a person having ordinary skill in the art as an Input/Output (I/O) module 460. The I/O module 460 regulates a plurality of inputs and outputs with regard to the computing device 400, wherein the inputs are a plurality of signals and data received by the computing device 400, and the outputs are the plurality of signals and data sent from the computing device 400. The I/O module 460 interfaces with a plurality of hardware, such as, but not limited to, non-volatile storage 461, communication devices 462, sensors 463, and peripherals 464. The plurality of hardware is used by at least one of, but not limited to, humans, the environment, and another computing device 400 to communicate with the present computing device 400. The I/O module 460 may comprise a plurality of forms, for example, but not limited to channel I/O, port mapped I/O, asynchronous I/O, and Direct Memory Access (DMA).

Consistent with the embodiments of the present disclosure, the aforementioned computing device 400 may employ a non-volatile storage sub-module 461, which may be referred to by a person having ordinary skill in the art as one of secondary storage, external memory, tertiary storage, off-line storage, and auxiliary storage. The non-volatile storage sub-module 461 may not be accessed directly by the CPU 420 without using an intermediate area in the memory 440. The non-volatile storage sub-module 461 may not lose data when power is removed and may be orders of magnitude less costly than storage used in memory 440. Further, the non-volatile storage sub-module 461 may have a slower speed and higher latency than in other areas of the computing device 400. The non-volatile storage sub-module 461 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (461) may comprise a plurality of embodiments, such as, but not limited to:
Optical storage, for example, but not limited to, Compact Disk (CD) (CD-ROM/CD-R/CD-RW), Digital Versatile Disk (DVD) (DVD-ROM/DVD-R/DVD+R/DVD-RW/DVD+RW/DVD±RW/DVD+R DL/DVD-RAM/HD-DVD), Blu-ray Disk (BD) (BD-ROM/BD-R/BD-RE/BD-R DL/BD-RE DL), and Ultra-Density Optical (UDO).
Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.
Magnetic storage such as, but not limited to, Hard Disk Drive (HDD), tape drive, carousel memory, and Card Random-Access Memory (CRAM).
Phase-change memory
Holographic data storage such as Holographic Versatile Disk (HVD).
Molecular Memory
Deoxyribonucleic Acid (DNA) digital data storage

Consistent with the embodiments of the present disclosure, the computing device 400 may employ a communication sub-module 462 as a subset of the I/O module 460, which may be referred to by a person having ordinary skill in the art as at least one of, but not limited to, a computer network, a data network, and a network. The network may allow computing devices 400 to exchange data using connections, which may also be known to a person having ordinary skill in the art as data links, which may include data links between network nodes. The nodes may comprise networked computer devices 400 that may be configured to originate, route, and/or terminate data. The nodes may be identified by network addresses and may include a plurality of hosts consistent with the embodiments of a computing device 400. Examples of computing devices that may include a communication sub-module 462 include, but are not limited to, personal computers, phones, servers, drones, and networking devices such as, but not limited to, hubs, switches, routers, modems, and firewalls.

Two nodes can be considered networked together when one computing device 400 can exchange information with the other computing device 400, regardless of any direct connection between the two computing devices 400. The communication sub-module 462 supports a plurality of applications and services, such as, but not limited to World Wide Web (WWW), digital video and audio, shared use of application and storage computing devices 400, printers/scanners/fax machines, email/online chat/instant messaging, remote control, distributed computing, etc. The network may comprise one or more transmission mediums, such as, but not limited to conductive wire, fiber optics, and wireless signals. The network may comprise one or more communications protocols to organize network traffic, wherein application-specific communications protocols may be layered, and may be known to a person having ordinary skill in the art as being improved for carrying a specific type of payload, when compared with other more general communications protocols. The plurality of communications protocols may comprise, but are not limited to, IEEE 802, ethernet, Wireless LAN (WLAN/Wi-Fi), Internet Protocol (IP) suite (e.g., TCP/IP, UDP, Internet Protocol version 4 [IPv4], and Internet Protocol version 6 [IPv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], Integrated Digital Enhanced Network [IDEN], Long Term Evolution [LTE], LTE-Advanced [LTE-A], and fifth generation [5G] communication protocols).

The communication sub-module 462 may comprise a plurality of size, topology, traffic control mechanisms and organizational intent policies. The communication sub-module 462 may comprise a plurality of embodiments, such as, but not limited to:
Wired communications, such as, but not limited to, coaxial cable, phone lines, twisted pair cables (ethernet), and InfiniBand.
Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Wherein cellular systems embody technologies such as, but not limited to, 3G,4G (such as WiMAX and LTE), and 5G (short and long wavelength).
Parallel communications, such as, but not limited to, LPT ports.
Serial communications, such as, but not limited to, RS-232 and USB.
Fiber Optic communications, such as, but not limited to, Single-mode optical fiber (SMF) and Multi-mode optical fiber (MMF).
Power Line communications

The aforementioned network may comprise a plurality of layouts, such as, but not limited to, bus networks such as Ethernet, star networks such as Wi-Fi, ring networks, mesh networks, fully connected networks, and tree networks. The network can be characterized by its physical capacity or its organizational purpose. Use of the network, including user authorization and access rights, may differ according to the layout of the network. The characterization may include, but is not limited to a nanoscale network, a Personal Area Network (PAN), a Local Area Network (LAN), a Home Area Network (HAN), a Storage Area Network (SAN), a Campus Area Network (CAN), a backbone network, a Metropolitan Area Network (MAN), a Wide Area Network (WAN), an enterprise private network, a Virtual Private Network (VPN), and a Global Area Network (GAN).

Consistent with the embodiments of the present disclosure, the aforementioned computing device 400 may employ a sensors sub-module 463 as a subset of the I/O 460. The sensors sub-module 463 comprises at least one of the device, module, or subsystem whose purpose is to detect events or changes in its environment and send the information to the computing device 400. Sensors may be sensitive to the property they are configured to measure, may not be sensitive to any property not measured but be encountered in its application, and may not significantly influence the measured property. The sensors sub-module 463 may comprise a plurality of digital devices and analog devices, wherein if an analog device is used, an Analog to Digital (A-to-D) converter must be employed to interface the said device with the computing device 400. The sensors may be subject to a plurality of deviations that limit sensor accuracy. The sensors sub-module 463 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:
Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator-semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nanosensors).
Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (o2), parking sensor, radar sensor, torque sensor, variable reluctance sensor, and water-in-fuel sensor.
Acoustic, sound and vibration sensors, such as, but not limited to, microphone, lace sensors such as a guitar pickup, seismometer, sound locator, geophone, and hydrophone.
Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.
Environmental, weather, moisture, and humidity sensors, such as, but not limited to, actinometer, air pollution sensor, moisture alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.
Flow and fluid velocity sensors, such as, but not limited to, air flow meter, anemometer, flow sensor, gas meter, mass flow sensor, and water meter.
Ionizing radiation and particle sensors, such as, but not limited to, cloud chamber, Geiger counter, Geiger-Muller tube, ionization chamber, neutron detection, proportional counter, scintillation counter, semiconductor detector, and thermoluminescent dosimeter.
Navigation sensors, such as, but not limited to, airspeed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.
Position, angle, displacement, distance, speed, and acceleration sensors, such as but not limited to, accelerometer, displacement sensor, flex sensor, free-fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.
Imaging, optical and light sensors, such as, but not limited to, CMOS sensor, colorimeter, contact image sensor, electro-optical sensor, infra-red sensor, kinetic inductance detector, LED configured as a light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photoswitch, phototube, scintillometer, Shack-Hartmann, single-photon avalanche diode, superconducting nanowire single-photon detector, transition edge sensor, visible light photon counter, and wavefront sensor.
Pressure sensors, such as, but not limited to, barograph, barometer, boost gauge, bourdon gauge, hot filament ionization gauge, ionization gauge, McLeod gauge, Oscillating U-tube, permanent downhole gauge, piezometer, Pirani gauge, pressure sensor, pressure gauge, tactile sensor, and time pressure gauge.
Force, Density, and Level sensors, such as, but not limited to, bhangmeter, hydrometer, force gauge or force sensor, level sensor, load cell, magnetic level or nuclear density sensor or strain gauge, piezocapacitive pressure sensor, piezoelectric sensor, torque sensor, and viscometer.
Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared/quartz/resistance thermometer, silicon bandgap temperature sensor, thermistor, and thermocouple.
Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 400 may employ a peripherals sub-module 464 as a subset of the I/O 460. The peripheral sub-module 464 comprises ancillary devices used to put information into and get information out of the computing device 400. There are 3 categories of devices comprising the peripheral sub-module 464, which exist based on their relationship with the computing device 400, input devices, output devices, and input/output devices. Input devices send at least one of data and instructions to the computing device 400. Input devices can be categorized based on, but not limited to:
Modality of input, such as, but not limited to, mechanical motion, audio, visual, and tactile.
Whether the input is discrete, such as but not limited to, pressing a key, or continuous such as, but not limited to the position of a mouse.
The number of degrees of freedom involved, such as, but not limited to, two-dimensional mice and three-dimensional mice used for Computer-Aided Design (CAD) applications.

Output devices provide output from the computing device 400. Output devices convert electronically generated information into a form that can be presented to humans. Input/output devices perform both input and output functions. It should be understood by a person having ordinary skill in the art that the ensuing are non-limiting embodiments of the aforementioned peripheral sub-module 464:
Input Devices
   Human Interface Devices (HID), such as, but not limited to, pointing device (e.g., mouse, touchpad, joystick, touchscreen, game controller/gamepad, remote, light pen, light gun, infrared remote, jog dial, shuttle, and knob), keyboard, graphics tablet, digital pen, gesture recognition devices, magnetic ink character recognition, Sip-and-Puff (SNP) device, and Language Acquisition Device (LAD).
   High degree of freedom devices, that require up to six degrees of freedom such as, but not limited to, camera gimbals, Cave Automatic Virtual Environment (CAVE), and virtual reality systems.
   Video Input devices are used to digitize images or video from the outside world into the computing device 400. The information can be stored in a multitude of formats depending on the user's requirement. Examples of types of video input devices include, but are not limited to, digital camera, digital camcorder, portable media player, webcam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.
   Audio input devices are used to capture sound. In some cases, an audio output device can be used as an input device to capture produced sound. Audio input devices allow a user to send audio signals to the computing device 400 for at least one of processing, recording, and carrying out commands. Devices such as microphones allow users to speak to the computer to record a voice message or navigate software. Aside from recording, audio input devices are also used with speech recognition software. Examples of types of audio input devices include, but not limited to microphone, Musical Instrumental Digital Interface (MIDI) devices such as, but not limited to a keyboard, and headset.
   Data AcQuisition (DAQ) devices convert at least one of analog signals and physical parameters to digital values for processing by the computing device 400. Examples of DAQ devices may include, but not limited to, Analog to Digital Converter (ADC), data logger, signal conditioning circuitry, multiplexer, and Time to Digital Converter (TDC).
Output Devices may further comprise, but not be limited to:
   Display devices may convert electrical information into visual form, such as, but not limited to, monitor, TV, projector, and Computer Output Microfilm (COM). Display devices can use a plurality of underlying technologies, such as, but not limited to, Cathode-Ray Tube (CRT), Thin-Film Transistor (TFT), Liquid Crystal Display (LCD), Organic Light-Emitting Diode (OLED), MicroLED, E Ink Display (ePaper) and Refreshable Braille Display (Braille Terminal).
   Printers, such as, but not limited to, inkjet printers, laser printers, 3D printers, solid ink printers, and plotters.
   Audio and Video (AV) devices, such as, but not limited to, speakers, headphones, amplifiers, and lights, which include lamps, strobes, DJ lighting, stage lighting, architectural lighting, special effect lighting, and lasers.
   Other devices such as Digital to Analog Converter (DAC)
Input/Output Devices may further comprise, but not be limited to, touchscreens, networking devices (e.g., devices disclosed in network sub-module 462), data storage devices (non-volatile storage 461), facsimile (FAX), and graphics/sound cards.

All rights, including copyrights in the code included herein, are vested in and the property of the Applicant. The Applicant retains and reserves all rights in the code included herein, and grants permission to reproduce the material only in connection with the reproduction of the granted patent and for no other purpose.

The following is claimed:

1. A case management platform comprising:
one or more hardware processors; and
one or more non-transitory computer-readable storage media storing instructions which, when executed by the one or more hardware processors, cause the one or more hardware processors to perform operations comprising:
connecting to one or more communication sources and one or more software systems to monitor for notifications;
receiving a notification comprising at least one of:
a communication from a stakeholder via the one or more communication sources, and
an event notification from the one or more software systems;
parsing the notification to determine information relevant to performing one or more actions;
based on the information contained in the notification, causing one or more actions to be performed, wherein the one or more actions comprise one or more of:
executing one or more operations in at least one software system of the one or more software systems,
transmitting a synthesized voice communication to one or more stakeholders associated with the notification, and
storing records associated with the notification to a database.

2. The case management platform of claim 1, wherein the operations further comprise:
responsive to receiving the notification, transmitting the parsed information to a machine learning engine;
receiving, from the machine learning engine, instructions for performing the one or more actions; and
performing the one or more actions based on the received instructions.

3. The case management platform of claim 2, wherein the machine learning engine comprises a large language model.

4. The case management platform of claim 1, wherein the one or more communication sources comprise at least one of a telephone system, an email system, a text messaging system, and a proprietary messaging application.

5. The case management platform of claim 1, wherein the one or more software systems comprise at least one of a scheduling system, a client management system, a caregiver management system, a billing system, an authorization system, a payroll system, a timecard system, and a compliance tracking system.

6. The case management platform of claim 1, wherein parsing the notification comprises:
performing speech-to-text processing on a voice communication to generate a text document; and
performing natural language processing on the text document to extract the information relevant to performing the one or more actions.

7. The case management platform of claim 1, wherein the operations further comprise:
generating a visual avatar configured to interact with the stakeholder; and
causing the visual avatar to present information to the stakeholder related to the one or more actions performed.

8. The case management platform of claim 1, wherein the operations further comprise:
determining that additional information is needed to perform the one or more actions;
generating a request for the additional information; and
transmitting the request to at least one of the stakeholder and a software system of the one or more software systems.

9. The case management platform of claim 1, wherein the stakeholder is one of a caregiver, a client, a client family member, a client guardian, an agency manager, and a payer.

10. The case management platform of claim 1, wherein the one or more actions comprise at least one of:
managing authorized caregiving hours for a client,
managing timecard exceptions,
advocating for a client,
coordinating care for a client,
coordinating benefits for a caregiver,
documenting client interactions,
ensuring caregiver work compliance,
managing a crisis, and
supervising staff.

11. A method for case management, the method comprising:
connecting to one or more external communication sources and one or more software systems to monitor for notifications;
receiving a notification comprising at least one of:
a communication from a stakeholder via the one or more external communication sources, and
an event notification from the one or more software systems;
parsing the notification to determine information relevant to performing one or more actions;
based on the information contained in the notification, causing one or more actions to be performed, wherein the one or more actions comprise one or more of:
executing one or more operations in at least one software system of the one or more software systems,
transmitting a synthesized voice communication to one or more stakeholders associated with the notification, and
storing records associated with the notification to a database.

12. The method of claim 11, further comprising:
responsive to receiving the notification, transmitting the parsed information to a machine learning engine;
receiving, from the machine learning engine, instructions for performing the one or more actions; and
performing the one or more actions based on the received instructions.

13. The method of claim 12, wherein the machine learning engine comprises a large language model.

14. The method of claim 11, wherein the one or more external communication sources comprise at least one of a telephone system, an email system, a text messaging system, and a proprietary messaging application.

15. The method of claim 11, wherein the one or more software systems comprise at least one of a scheduling system, a client management system, a caregiver management system, a payroll system, a timecard system, and a compliance tracking system.

16. The method of claim 11, wherein parsing the notification comprises:
performing speech-to-text processing on a voice communication to generate a text document; and
performing natural language processing on the text document to extract the information relevant to performing the one or more actions.

17. The method of claim 11, further comprising:
generating a visual avatar configured to interact with the stakeholder; and
causing the visual avatar to present information to the stakeholder related to the one or more actions performed.

18. The method of claim 11, further comprising:
determining that additional information is needed to perform the one or more actions;
generating a request for the additional information; and
transmitting the request to at least one of the stakeholder and a software system of the one or more software systems.

19. The method of claim 11, wherein the stakeholder is one of a caregiver, a client, a client family member, a client guardian, an agency manager, and a payer.

20. One or more non-transitory computer-readable storage media storing instructions which, when executed by one or more hardware processors, cause the one or more hardware processors to perform operations comprising:
connecting to one or more external communication sources and one or more software systems to monitor for notifications;
receiving a notification comprising at least one of:
a communication from a stakeholder via the one or more external communication sources, and
an event notification from the one or more software systems;
parsing the notification to determine information relevant to performing one or more actions;
based on the information contained in the notification, causing one or more actions to be performed, wherein the one or more actions comprise one or more of:
executing one or more operations in at least one software system of the one or more software systems,
transmitting a synthesized voice communication to one or more stakeholders associated with the notification, and
storing records associated with the notification to a database.
